(19) United States
(12) Patent Application Publication
(10) Pub. No.: US 2007/0066996 A1
(43) Pub. Date: Mar. 22, 2007

(54) Modafinil-based neurorehabilitation of impaired neurological function associated with brian injury

(76) Inventors: Daniel E Katzman, Newton, MA (US); Elkan R. Gamzu, Newton, MA (US); Neal M. Farber, Waban, MA (US); Esteban A. Fridman, Capital Federal (AR)

Correspondence Address: Leon R Yankwich;Yankwich & Associates, 201 Broadway, Cambridge, MA, 02139, US

(21) Appl. No.: 10/549,488

(22) PCT Filed: Mar. 17, 2004
(86) PCT No.: PCT/US04/08056

§ 371 Date: Jul. 31, 2006

Related U.S. Application Data
(60) Provisional application No. 60/455,405, filed on Mar. 17, 2003.

(51) Int. Cl. A61N 1/00

(52) U.S. Cl. 607/3

(57) ABSTRACT

The invention provides methods of treating an impaired neurological function associated with brain injury in an individual comprising administering to the individual an effective amount of modafinil. The use of modafinil, especially in conjunction with a neurorehabilitation program, is effective to improve or restore one or more neurological functions that were impaired due to brain injury in an individual.

### CROSS-REFERENCE TO RELATED APPLICATION

This application claims priority to U.S. Provisional Application No. 60/455,405, filed Mar. 17, 2003.

### FIELD OF THE INVENTION

The present invention relates to the fields of neurology and neurorehabilitation. In particular, this invention is related to treatments to restore impaired neurological function associated with brain injury in an individual.

### BACKGROUND OF THE INVENTION

Currently, 10 million Americans live with long-term disabilities caused by stroke or traumatic brain injury (TBI). As a result of these events, patients are left with irreversible, debilitating losses of function that require ongoing, costly rehabilitation.

Traumatic brain injury (TBI) results from a physical impact to the head. Each year there are 2 million TBI incidents in the U.S., most of which are considered minor. However, about 500,000 to 750,000 of these incidents result in hospitalization. Of these, over 350,000 will recover and join the pool of over 4.5 million existing TBI patients that require some form of ongoing rehabilitation. Currently, there is no approved pharmaceutical treatments to aid these patients who often require many years of physical rehabilitation. In fact, four years after the initial injury, 15% of TBI victims are left with disabilities that prevent them from working and 10% of these patients are left with permanent disabilities.

TBI disproportionately affects young people ages 15 to 24 years old (mostly external head injuries). Disability in this age group places an enormous financial and emotional burden on both society and a patient's family. Another group that is disproportionately represented are people over 60 years old who fall and sustain head injuries. The cost of rehabilitation of TBI victims is estimated to be $31 billion per year in the U.S. alone. These demographics translate into higher numbers of individuals that are at risk of TBI.

Stroke is a type of ischemic event in the brain in which a blood clot blocks a blood vessel or artery or in which a blood vessel breaks resulting in an interruption in blood flow to an area of the brain. Each year in the U.S., there are 750,000 new incidents of acute stroke, and stroke is the third leading cause of death in the U.S. There are nearly 5 million patients living in the U.S. with the after effects of stroke who require rehabilitation in order to increase motor control functionality. These patients may require many years of expensive physical rehabilitation.

In addition to the emotional toll on the families of stroke patients, the financial cost of stroke is estimated to be $30 billion per year in the U.S. alone. Although some strokes may be preventable, as in the case of TBI the population of stroke patients is growing mainly due to the increase in the aging population as well as the common occurrence in the U.S. of a sedentary lifestyle and poor diet. As with TBI, there is currently no cure to treat the damage caused by stroke. Current medical approaches focus on emergency treatments to maintain essential bodily functions. After the initial treatment, stroke victims may face years of rehabilitation or even permanent disabilities.

Due to constant advances in effective emergency medicine, many patients that sustain a brain injury due to any of a variety of trauma or causes will survive, but they will remain in a severe altered state of consciousness, such as a coma, the deepest state of unconsciousness, or another more emergent, but altered state of consciousness, such as persistent vegetative state or minimally conscious state. Such patients fail to emerge to the fully functional state of awareness of self and environment that they possessed prior to brain injury. Occasionally, some patients may emerge from a particular, deeper state of altered consciousness to a higher state, or even to normal awareness. However, even with emergence to full awareness, it is not uncommon for such individuals to require some form of neurorehabilitation to improve or regain any of a number of neurological functions, such as communication skills, motor skills, memory skills, and various other cognitive functions that permit self care, mobility, and employability. Neurorehabilitative programs have the goal of restoring in an individual any of a variety of neurological functions that may have been impaired due to a brain injury. By way of example, such neurological functions may include communication skills (speaking, writing), cognitive skills (e.g., reasoning, memory), and motor skills (directed movements, walking, running, balancing). Progress in restoring or improving one or more impaired neurological functions using current neurorehabilitative programs may be quite slow; yet progress to restoring function can be achieved to varying extents in many instances.

Interestingly, in recent years, important new findings have been made that indicate an ability of the neural network of the brain of a trauma or stroke patient to reorganize itself, a mechanism known as neural plasticity or adaptive plasticity, in which interactions between surviving neurons may adopt a new function or be recruited to restore a lost neurological function. A treatment for impaired neurological function that is directed at surviving neurons and neurotransmission may expedite the neurorehabilitation process.

With approximately 10 million TBI and stroke patients in need of rehabilitation and an additional 1 million or more entering the patient pool annually in the U.S., there is a clear need for a pharmaceutical therapy to aid the rehabilitation of such individuals. Clearly, needs remain for effective therapies to treat impaired neurological function in victims of brain injury.

### SUMMARY OF THE INVENTION

The present invention addresses the above problems and needs by providing methods and means for treating one or more impaired neurological functions associated with brain injury in an individual comprising administering to the individual modafinil (benzhydrylsulfinylacetamide). Impaired neurological functions treated according to the invention are those associated with an injury to the brain of an individual, such as may arise from any of a variety of events or disorders, including, but not limited to, traumatic brain injury (TBI, e.g., from a fall on a hard surface, vehicle accident, strike to the head), an ischemic event (e.g., stroke), an anoxic event, a hypoxic event, an anoxic-ischemic encephalopathy (e.g., brain injury associated with cardiovascular bypass surgery), spinal cord injury, major organ failure, a drug-induced brain injury (e.g., anesthesia-induced, illicit drug use), encephalitis, multiple sclerosis, and degenerative diseases (e.g., Parkinson's Disease).

Impaired neurological functions associated with brain injury that may be treated with methods and compositions according to the invention include, but are not limited to, those that are primarily cognitive functions (e.g., reading, memory, voice recognition), primarily sensory functions (e.g., tactile sensing, hot-cold sensing, light sensing), primarily motor functions (e.g., strength, direction, speed involved in body movements, such as, walking, running, maintaining balance), or a combination of such functions (e.g., coordination of cognitive and motor functions, as required in speaking, writing, use of tools, operating machines, and other activities).

In a preferred embodiment, modafinil is administered to an individual according to the invention at a dose in the range of from 50 to 600 mg per day, and, more preferably, 200 mg/day.

In another embodiment, the invention provides methods for treating one or more impaired neurological functions associated with a brain injury in an individual comprising administering to the individual an effective amount of modafinil in conjunction with (e.g., co-administration, concurrent administration, sequential administration) one or more additional compounds that may provide a beneficial pharmacological activity or property. In one embodiment, such an additional compound may be a dopaminergic agent, such as apomorphine, bromocriptine, amantadine, pergolide, pramipexole, ropinirole, fenoldopam, cabergoline, rotigotine, lysuride, talipexale, 7-OH DPAT, quinpirole, SKF-38393, L-dopa (levadopa), or combinations thereof. In another embodiment, modafinil may be administered in conjunction with a neurostimulant compound such as caffeine, an amphetamine, a dextroamphetamine, a methylphenidate, or combinations thereof

In another embodiment, the invention provides a method of treating one or more impaired neurological functions in an individual who has sustained a brain injury comprising administering to the individual an effective amount of modafinil in conjunction with any of a variety of neurorehabilitation programs for restoring neurological function. Neurorehabilitation programs useful in the invention include, without limitation, physical/sensory type protocols (exercises, tasks, light stimulation, audio stimulation, pictures, tactile stimulation), electric and/or magnetic stimulation regimens (e.g., transcranial magnetic stimulation (TMS), deep brain stimulation (DBS), electroconvulsive therapy), drug-based stimulation regimens (e.g., caffeine, amphetamines), and combinations thereof.

Modafinil may be administered orally, e.g., as tablets or pills, to an individual or using any of a variety of routes and means available and known in the art.

### DETAILED DESCRIPTION

The present invention provides methods and compositions to treat impaired, i.e., diminished or lost, one or more neurological functions in an individual who has sustained a brain injury, including traumatic brain injury and stroke, comprising administering to the individual an effective amount of modafinil (benzhydrylsulfinylacetamide). Methods of the invention also comprise administering modafinil to an individual in conjunction with a neurorehabilitation program. An impaired neurological function of an individual may be significantly improved and/or accelerated when modafinil is administered to the individual in conjunction with the individual undergoing a rehabilitation program.

In order that the invention may be more clearly understood, the following terms are used as defined below.

A “drug” refers to any compound or composition that has a pharmacological activity. Thus, a “therapeutic drug” is a compound or composition that can be administered to an individual to provide a desired pharmacological activity to the individual for treating an undesired or harmful disorder or condition, including, but not limited to, neurological impairments or disorders. A “prophylactic drug” is a compound or composition that can be administered to an individual to prevent or provide protection from the development in an individual of an undesired or harmful disorder or condition. A drug may have prophylactic as well as therapeutic uses. An “illicit drug” refers to a drug that is generally illegal to possess and/or use under any circumstances in a particular jurisdiction without governmental authority and includes illegal “recreational” and “addictive” compounds and controlled substances such as various opiates and psychotropic substances.

The term “brain injury” is a general term used to refer to a condition that results in central nervous system damage, irrespective of the physiopathological source. The most frequent origins of brain injury include stroke, traumatic brain injury (TBI), spinal cord injury, encephalitis, multiple sclerosis, major organ failure, and degenerative diseases (e.g., Parkinson's Disease).

“Neurological function” refers to a function of the body of an individual that requires normal functioning neural transmission. Neurological functions of an individual that may be impaired by brain injury, and, therefore treated according to the invention, include, without limitation, functions that are primarily sensory (e.g., light sensing, tactile sensing, hot-cold sensing), primarily cognitive (e.g., reading, memory, comprehension, reasoning), functions that are primarily based on motor activity (e.g., directed body movements, walking, maintaining balance), or a combination thereof (e.g., coordination of cognitive and motor functions as required in speaking, writing, use of tools, operating machines, and other activities). Impaired neurological functions may also be referred to by the name for the corresponding neurological deficit or disorder, e.g., aphasia, dysarthria, amnesia, paralysis, anesthesia, propioceptive deficits, and the like. Any of a variety of disorders or conditions may lead to the impairment of one or more neurological functions of an individual.

Traumatic brain injury (TBI) and stroke are among the most frequently occurring and widely known events that can cause brain injury and an associated impairment of one or more neurological functions. Among the variety of cases of TBI diagnosed each year in the United States and around the world are vehicle accidents, such as involving a car, motorcycle, or bicycle. Stroke represents the leading cause of disability in adulthood. Patients that suffer a stroke can present disabilities associated with impairment of any of a variety of neurological functions as described above, including, but not limited to, motor function (e.g., impairments in strength, dexterity, swallowing), sensory functions (e.g., anesthesia, propioceptive deficits), speech function (e.g., aphasia, dysarthria), and cognitive functions (e.g., deficiency in planning, short and long term memory loss (amnesia), working memory loss, attention deficits, spatial attention deficits).

“Neurorehabilitation”, as used herein, refers to any rehabilitation program that may be used for the purpose of improving, regaining, or restoring one or more neurological functions that may have been impaired (i.e., lost or diminished) in an individual as the result of a brain injury. Neurorehabilitation programs useful in the invention provide one or more neurostimuli designed to restore or enhance one or more impaired neurological functions of an individual. Such neurorehabilitation programs include programs that provide forms of physical therapy, occupational therapy, speech therapy, and various combinations thereof Examples of neurorehabilitation programs that may be used in conjunction with administering modafinil as described herein include, without limitation, physical/sensory type protocols (exercises, tasks, light stimulation, audio stimulation, visual stimulation, tactile stimulation), electrical/magnetic stimulation regimens (e.g., trans-cranial magnetic stimulation (TMS), deep brain stimulation (DBS), electroconvulsive therapy; see, also, U.S. Pat. No. 6,463,328), and/or drug-based stimulation regimens (e.g., caffeine, amphetamines, dopaminergic agent). For example, a neurorehabilitation program may comprise having an individual who has sustained a brain injury perform or attempt to perform, often in multiple repetitions, one or more particular exercises or tasks designed to improve or restore one or more neurological functions. Thus, such exercises or tasks may include forms of physical therapy to promote development of an impaired motor function; exercises or tasks for improving aspects of cognitive functions as well, e.g., memory, reading, recognition of objects, comprehension, response to commands, and the like; and exercises or tasks designed to improve a combination of motor and cognitive functions, e.g., speech, writing, operating machines, and the like. The goal of neurorehabilitation is to improve or restore one or more neurological functions that were impaired due to brain injury in an individual and, thereby, advance the individual toward increased participation and independence in self-care, mobility, and/or employment.

The term “modafinil” is synonymouns with benzhydrylsulfinyl acetamide and 2-[(diphenylmethyl)sulfinyl]acetamide as described in U.S. Pat. Nos. 5,612,379 and 6,489,363 (the teachings of both of which are incorporated herein by reference) and can be represented by the following formula for the neutral (free) base form of the compound: 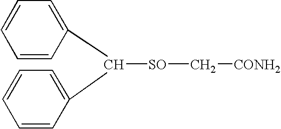
Modafinil has different enantiomers and thus may exist as a racemic mixture. If necessary, individual isomers may be resolved by methods known in the art (see, e.g., Donovan et al., Ther. Drug Monit., 25(2): 197-202 (2003)). It is also understood that the terms “modafinil”, “benzhydrylsulfinyl acetamide”, and “2-[(diphenylmethyl)sulfinyl]acetamide”encompass the various organic and inorganic acid salt forms of the above structure. Preparations and modes of delivery of modafinil are known in the treatment of other disorders (see, e.g., U.S. Pat. No. 5,612,379; U.S. Pat. No. RE37,516 E). In addition, methods and compositions of the invention comprising “modafinil”, as described above, may alternatively comprise, instead of modafinil, a pro-drug of modafinil, i.e., a compound that is metabolized to modafinil when administered to an individual. Modified modafinil compounds, derivates, analogues and mimics of modafinil are also known in the art, and functionally equivalent such compounds to modafinil may also be used in accordance with the teachings herein.

The precise pharmacological mechanism of action of modafinil is unclear (see, e.g., Physician's Desk Reference, 58th ed. (Thomson, Montvale, N.J., 2004), p. 1160). For example, one paper has suggested that modafinil modulates the central postsynaptic alpha-adrenergic receptor without participation of the dopaminergic system (Duteil et al., Eur. J. Pharmacol., 180: 49-58 (1990)). However, another study has reported that modafinil increased extracellular dopamine and that dopamine transporter knock-out mice were unresponsive to the action of modafinil (Wisor et al., J. Neuroscience, 21(5): 1787-1794 (2001)). The neuropsychopharmacological profile of modafinil has been distinguished from amphetamines (see, e.g., Saletu et al., Int. J. Clin. Pharm. Res., 9:183-195 (1989)).

“Neural plasticity”, “adaptive neural plasticity”, “adaptive plasticity”, and similar terms refer to the property of various surviving neurons or neural pathways after brain injury to be adopted or recruited to restore previously impaired neurological function. While not intending to be bound by any particular mechanism, the methods of treating impaired neurological function comprising administration of modafinil, as described herein, may be seen as effective in view of the concept of adaptive plasticity. Adaptive neural plasticity constitutes the basis for functional recovery in patients who have suffered brain damage. Neural plasticity appears to occur predominantly in cortical areas that are involved in specific neurological functions (e.g., primary motor cortex or premotor cortex in motor recovery, Broca in the case of aphasia, posterior parietal cortex in the case of sensory deficit). There is evidence indicating that this functional reorganization (adaptive plasticity) is accompanied by an increase in cortical excitability that would be modulated by GABAergic intracortical circuits. Moreover, those cortical changes need a process of consolidation in which the dopamineric system is essential. In the case of motor deficits, studies using transcranial magnetic stimulation (TMS) have demonstrated a correlation between the functional improvement and the enlargement of motor evoked potentials (MEP), the reduction of intracortical inhibition (ICI), and the increment of intracortical facilitation (ICF); three neurophysiological parameters of cortical excitability. While not intending to be bound by any particular theory or mechanism, modafinil may be viewed as a drug with the capability for modifying these intracortical circuits by means of modification of the most important neurotransmitters systems involved in recovery, the noradrenergic, the dopaminergic, and the GABAergic systems. In such a case, modafinil would appear to act as a dopaminergic agent, a GABAergic antagonist, and as a noradrenergic agent, as well as having all the properties to produce improvement in performance in convalescent neurological patients.

Phrases that refer to administering or the administration of modafinil to an individual “in conjunction with” another drug, composition, or procedure (or vice versa) as described herein are understood to refer to any combination of therapeutic methods, compositions, or procedures that encompasses co-administration (i.e., administration together, e.g., as in a solution, dispersion, or other mixture), concurrent administration (essentially at the same time), or sequential administration (before or after) of the other drug, composition, or rehabilitative procedure (e.g., task or exercise for cognitive and/or motor function) in addition to the administration of modafinil. It is also understood that administration of a drug or other composition to an individual “in conjunction with” modafinil according to the invention may comprise using the same or different route used to administer the modafinil to an individual.

By “pharmaceutically acceptable” is meant a material that is not biologically, chemically, or in any other way, incompatible with body chemistry and metabolism and also does not adversely affect the desired, effective activity of modafinil or any other component in a composition that may be administered to an individual according to the invention.

Terms such as “parenteral”, “parenterally”, and the like, refer to routes or modes of administration of a compound or composition to an individual other than along the alimentary canal. Examples of parenteral routes of administration include, without limitation, subcutaneous (s.c.), intravenous (i.v.), intramuscular (i.m.), intra-arterial (i.a.), intraperitoneal (i.p.), transdermal (absorption through the skin or dermal layer), nasal or pulmonary (e.g., via inhalation or nebulization, for absorption through the respiratory mucosa or lungs), direct injections or infusions into body cavities or organs, as well as by implantation of any of a variety of devices into the body (e.g., of a composition, depot, or device that permits active or passive release of a compound or composition into the body). One or more parenteral routes of administration may be employed in methods described herein.

The terms “enteral”, “enterally”, “oral”, “orally”, “non-parenteral”, “non-parenterally”, and the like, refer to administration of a compound or composition to an individual by a route or mode along the alimentary canal. Examples of enteral routes of administration include, without, limitation, oral, as in swallowing solid (e.g., tablet) or liquid (e.g., syrup) forms; sub-lingual (absorption under the tongue); nasojejunal or gastrostomy tubes (into stomach); intraduodenal administration; as well as rectal administration (e.g., suppositories for release and absorption of a compound or composition by in the lower intestinal tract of the alimentary canal). One or more enteral routes of administration may be employed in methods described herein.

The meaning of other terms will be evident by the context of use and, unless otherwise indicated, are consistent with the meanings understood by those skilled in the fields of neurology and neurorehabilitation.

Assessment of Neurological Functions

The neurological health and functions of an individual who has sustained a brain injury may be assessed and/or monitored by a neurologist or other skilled healthcare professional that observes a change in the efficiency and/or accuracy of an individual in performing or attempting to perform some exercise or task requiring one or more neurological functions. A variety of such tasks are used in rehabilitation programs including, but not limited to, organizing or partitioning objects from a mixture, such as sorting objects or placing pegs in a pegboard; strength tests, such as squeezing force; walking; speaking; range of motion of limbs; and response to oral, audio, or visual stimuli.

In addition to rehabilitation programs, a number of scales are known that typically provide a defined set of parameters or tasks that are conducted or administered by a trained practitioner to assess the neurological functions of an individual that has sustained a brain injury. An example of such a scale is the Disability Rating Scale (DRS).

The DRS (Rappaport et al., Arch. Phys. Med. Rehabil., 63; 118-123 (1982)) was originally developed and tested with older juvenile and adult individuals with moderate and severe traumatic brain injury. This scale may be used to track an individual from coma to re-integration into the community. Various items in this scale address impairment, disability, and handicap. The DRS is a 31-point scale ranging from 0 (no disability) to 30 (death). Accordingly, the maximum score a living patient can obtain is 29 (extreme vegetative state) and 1 to 28 represent different grades of disability. A disadvantage of this scale is that it is relatively insensitive at the low end of the scale (i.e., mild traumatic brain injury). In particular, the scale does not have the ability to reflect very subtle, but sometimes significant, changes in an individual within a specific window of recovery.

The Functional Independence Measure™ (FIM) assessment scale (Guide for the Uniform Data Set for Medical Rehabilitation (including the FIM™ instrument), Version 5.1 (State University of New York at Buffalo, Buffalo, N.Y., 1997)) is the most widely accepted functional assessment measure currently in use in the field of rehabilitation. The FIM™ is an 18-item ordinal scale that may be employed with all diagnoses within a rehabilitation population. It is viewed as particularly useful for assessment of progress during inpatient rehabilitation (Functional Assessment and Outcome Measurement for the Rehabilitation Health Professional, Dittmar, S. and Gresham, G. E., eds. (Aspen Press, 1997)).

The most desired outcome of applying a treatment as described herein to an individual who has sustained a brain injury is restoration of all neurological functions at least to the level that existed prior to brain injury.

Therapeutic Methods and Compositions

The methods of the invention for treating an impaired neurological function in an individual who has sustained a brain injury comprise administering to the individual an effective amount of modafinil. Modafinil may be administered as the sole therapeutic agent or in conjunction with (e.g., co-administration, concurrent administration, sequential administration) one or more additional compounds that may provide an additional, desirable beneficial pharmacological activity or property. Modafinil is typically administered orally in tablet form (e.g., marketed as PROVIGIL®, Cephalon, West Chester, Pa.). It is understood that any additional therapeutic compound that is to be included in a method described herein may be administered to an individual in a mixture with or separate from modafinil and by the same or different route as used for administering modafinil. Examples of such additional compounds that may be administered in conjunction with modafinil according to the invention are neurostimulant compounds and dopaminergic agents.

Dosing for a particular individual (patient) who has sustained a brain injury will be determined by the attending neurologist or other skilled healthcare provider taking into account a variety of clinical parameters that characterize that patient, e.g., state of consciousness, overall neurological condition, other injuries, cardiovascular condition, age, gender, weight, possible genetic factors, and the like. Preferably, modafinil is administered to an individual at a dose in the range of from 50 to 600 mg/day. Thus, doses of 100, 200, 400, and 600 mg/day may be used in the methods described herein. A particularly useful dose to initiate treatment and which may also be maintained during a course of treatment is 200 mg/day of modafinil. Furthermore, modafinil may be administered periodically or cyclically to an individual, e.g., administration to an individual for a period of time, discontinued for a period for time, and then re-initiated. The limitation on a course of dosing or repetition of dosing typically will be based on whether the attending healthcare believes such dosing or repetition may or may not provide further benefit to an impaired neurological function and/or whether there is any evidence of acute side effects that would limit the use of a particular dose or duration of administering modafinil to an individual.

It is also understood that persons skilled in the art are aware that doses of pharmacologically active compounds, such as modafinil, may be expressed not only in terms of mass (e.g., mg) of drug administered per day, but other units as well as, including, but not limited to, mg per kilogram (kg) of body mass, mg per surface area, mg per unit volume of formulation, and the like. As used herein, discussion of dosages in terms of mg/day refer to mg per patient per day and are based on the commonly used standard of a 70 kg male human patient. Similarly, discussion of dosing in terms of mg of compound per kg of body weight (mass) assume a 70 kg male human being. Hence, it is understood that when treating an individual that is more or less than 70 kg a dose may be appropriately modified in accordance with standard pharmacological adjustments. Thus, various examples of doses described herein are readily converted by persons skilled in the art to various other dosing units (and vice versa) required for treating specific individuals with particular pharmaceutically acceptable formulations.

Pharmaceutical preparations and routes of administration useful in the invention may generally be adapted from what is already known for administering modafinil in existing therapies, such as for treating hypersonmia and narcolepsy (see, e.g., Bastuji et al., Prog. Neuro-Psych. & BioL Psych., 12: 695-700 (1988)). Other factors that may be considered by the clinician in administering modafinil to an individual include, but are not limited to, the state of consciousness, the loss or degradation of one or more neurological functions, age, weight, sex, and possible genetic factors.

Modafinil may be administered to an individual in conjunction with a neurostimulant compound such as caffeine, an amphetamine, a dextroamphetamine, a methylphenidate, or combinations thereof.

Modafinil may also be administered to an individual in conjunction with a dopaminergic agent. Preferably, when administered, the dopaminergic agent crosses the blood-brain barrier. A variety of dopaminergic agents are known that may be administered in conjunction with modafinil according to the invention, including, without limitation, apomorphine, bromocriptine, amantadine, pergolide, pramipexole, ropinirole, fenoldopam, cabergoline, rotigotine, lysuride, talipexale, 7-OH DPAT, quinpirole, SKF-38393, L-dopa (levadopa), or combinations thereof. In fact, it has been discovered that the high potency dopamine agonist apomorphine or high doses of the dopamine precursor L-dopa are particularly effective at treating impaired neurological function, including emergence from coma and other altered consciousness states, in individuals who have sustained a brain injury (see, commonly owned, co-filed, international application No. PCT/US04/______; [Atty. Docket No. NEU-101.1 PCT).

Modafinil may also be administered to an individual in conjunction with a neurorehabilitation program that is designed to stimulate neural connections in order to restore or improve one or more impaired neurological functions. When administered in conjunction with a neurorehabilitation program, modafinil may be administered prior to administering the neurorehabilitation program, as well as, continued throughout the time period over which the neurorehabilitation program is continued. For example, modafinil may be administered to an individual prior to having the individual perform or attempt to perform an exercise or task designed for improving or restoring a neurological function of the individual. Moreover, the administration of modafinil in conjunction with a neurorehabilitation program may be applied one or more times to the same individual to provide therapeutic cycles. Preferably, when one or more such modafinil-based therapeutic cycles are employed, the individual is provided a period of rest from modafinil administration and participation in a neurorehabilitation program. For example, a therapeutic cycle may comprise administering modafinil to an individual for a period of two weeks prior to or simultaneously with the administration of a neurorehabilitation program. The individual may then be allowed a rest for a period of time, e.g., 4 to 12 weeks, free of administration of modafinil and the neurorehabilitation program. After such a rest period and, preferably, after an assessment of neurological function, an attending healthcare provider may decide to re-initiate a treatment period comprising administering to the individual modafinil in conjunction with a neurorehabilitation program to provide further improvement in one or more neurological functions of the individual. It is understood that the dose of modafinil and the neurorehabilitation program administered to an individual during any treatment period maybe the same or different from those used in any preceding treatment period as deemed necessary or appropriate by the attending healthcare provider for treating one or more impaired neurological functions.

Moreover, it is understood that the healthcare provider determines the proper time to commence treatment for impaired neurological function according to the invention, taking into account various factors for any particular patient who has sustained a brain injury. For example, in the case of a stroke patient, a neurorehabilitation program is, as a general rule, not commenced until after the patient has been medically stabilized from the acute effects of the stroke, i.e., after the attending healthcare provider has determined that the patient is no longer presenting evidence of or in danger of further significant physiological deterioration warranting the need to apply an acute therapeutic regimen (e.g., to stop bleeding, stabilize cardiovascular function, control of seizures, and the like).

Neurorehabilitation programs useful in the invention may include, without limitation, physical/sensory type protocols, electrical/magnetic stimulation regimens, and/or drug-based stimulation regimens. Physical/sensory stimulation programs or protocols available in the art are particularly useful in the invention and may include any of the well-known methods employed in clinical neurology and neurorehabilitation to stimulate a response by one or more of the five senses. Such methods may include applying, without limitation, one or more sensory stimuli such as light, color, a visual scene (e.g., a picture), hot or cold temperature, tactile stimulation (e.g., for surface feeling), a smell, a taste, a sound (e.g., a voice of a family member), and the like.

In addition, various electrical/magnetic methods are now available that provide electric or magnetic stimulation to the brain. Such methods, which may be used in conjunction with administering modafinil include, but are not limited to, vagal nerve stimulation, cranial nerve stimulation by electrical pulse waveform, neuromodulation using a pulsed electrical stimulus, electroconvulsive therapy, trans-cranial magnetic stimulation (TMS), deep brain stimulation (DBS), and the like.

An example of a useful neurorehabilitation regimen according to the invention comprises administering modafinil at a dose in the range of from 50 to 600 mg per day, such as 200 mg of modafinil per day, to an individual who has sustained a brain injury 1 to 3 hours prior to engaging in (or attempting such) one or more tasks or exercises of a neurorehabilitation program. Preferably, dosing and tasking are carried out at least five days per week for at least two weeks or longer. Depending on the individual, it may be useful to perform or attempt to perform tasks or exercises more than once per day.

As noted above, modafinil is commercially available in tablet form for oral administration (e.g., marketed as PROVIGIL®, Cephalon, West Chester, Pa.). More generally, compositions useful in the invention may be formulated for administration to an individual according to standard pharmaceutical protocols and texts (e.g., Remington's Pharmaceutical Sciences, 18th ed., Alfonso R. Gennaro, ed. (Mack Publishing Co., Easton, Pa. 1990)). The pharmaceutical compositions of this invention for oral administration may include, but are not limited to, tablets, pills, capsules, caplets, aqueous solution, oleaginous suspensions, syrups, or elixirs. In the case of tablets for oral use, carriers, which are commonly used include lactose and corn starch. Lubricating agents, such as magnesium stearate, may also be added. Capsules, tablets, pills, and caplets may also be formulated for delayed or sustained release. If desired, certain sweetening and/or flavoring and/or coloring agents may also be added.

Thus, a composition comprising modafinil may also comprise any of a number of various pharmaceutically acceptable buffers (carriers), excipients, or adjuvants known in the art that may provide one or more beneficial pharmacological properties, including but not limited to, more efficient or less painful administration to an individual, more efficient or time-released delivery of modafinil to the central nervous system, and/or longer storage of compositions (i.e., enhanced shelf-life). Accordingly, pharmaceutical compositions of this invention may include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions employed in the invention may be in the form of a sterile injectable preparation, such as a sterile injectable aqueous solution or an oleaginous suspension. Suspensions may be formulated according to techniques known in the art using suitable dispersing or wetting agents (e.g., an anionic detergent). A sterile injectable preparation may also be a sterile injectable solution or suspension in a non-toxic, parenterally acceptable diluent or solvent, such as a solution in 1,3-butanediol. Pharmaceutically acceptable aqueous buffer solutions that may be employed for parenteral administration of a compound or composition described herein include, without limitation, sterile water, physiological saline, bacteriostatic saline (e.g., saline containing about 0.9% benzyl alcohol), phosphate-buffered saline, Hank's solution, Ringer's-lactate and the like. In addition, sterile, fixed oils have been conventionally employed as a solvent or suspending medium for use in administering compositions. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

For application topically, a composition of the invention may be formulated for administration by a transdermal patch or similar device. Topical formulations may be prepared with a suitable ointment, gel, cream, or lotion containing the active components suspended or dissolved in a carrier. Carriers for topical administration include, but are not limited to, water, mineral oil, liquid petroleum, white petroleum, propylene glycol, polyoxyethylene polyoxypropylene compound, and emulsifying wax. One or more emollients may be present to enhance penetration through the skin. Other suitable carriers may include, but are not limited to, mineral oil, sorbitan monostearate, polysorbate 60, cetyl esters wax, cetearyl alcohol, 2-octyldodecanol, benzyl alcohol and water.

Compositions of this invention may also be administered in the form of suppositories for rectal administration. Such compositions can be prepared by mixing various desired pharmacologically active components, such as modafinil and other pharmacologically active agents, with a suitable non-irritating excipient, which is solid at room temperature but liquid at body temperature and, therefore, will melt in the rectum space to release the active components that can be absorbed across the gut wall. Such materials include, but are not limited to, cocoa butter, beeswax and polyethylene glycols.

The pharmaceutical compositions of this invention may be administered nasally or inhaled through the mouth in which case absorption of modafinil may occur via the mucus membranes of the respiratory tract, including the nose and/or lungs. Such modes of administration typically require that the composition be provided in the form of a powder, solution, or liquid suspension, which is then mixed with a gas (e.g., air, oxygen, nitrogen, etc., or combinations thereof) so as to generate an aerosol or suspension of droplets or particles. Such compositions are prepared according to techniques known in the art of pharmaceutical formulation and may be prepared as solutions in saline, employing, e.g., benzyl alcohol or other suitable preservatives, and/or other solubilizing or dispersing agents known in the art.

Pharmaceutical compositions of the invention may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administrations from the same container. Kits, of one or more doses, may be provided comprising modafinil in a pill or other form for oral delivery along with instructions for administering modafinil to treat an impaired neurological function. Alternatively, a kit may comprise a modafinil-containing composition in dry powder or lyophilized form, as well an appropriate diluent, which are to be combined shortly before administration.

Various antimicrobial agents may also be used in compositions of the invention to prevent degradation and contamination. Such commonly used antimicrobial agents included phenol, benzyl alcohol, meta-cresol, methyl paraben, propyl paraben, benzalconium chloride, and benzethonium chloride. Such agents are present at concentrations that will prevent the growth of bacteria, fungi, and the like, but be non-toxic when administered to the intended patient.

Consistent with good manufacturing practices, which are in current use in the pharmaceutical industry and which are well known to the skilled practitioner, all components contacting or comprising modafinil must be sterile and periodically tested for sterility in accordance with industry standards. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation. Choice of method of sterilization will be made by the skilled practitioner with the goal of effecting the most efficient sterilization that does not significantly alter a desired pharmacological activity of modafinil or any other component of a composition intended for administration to an individual. Ultrafiltration is a particularly useful method of sterilization for pharmaceutical compositions that are aqueous solutions or suspensions.

In order to more fully understand the invention, the following non-limiting examples are provided.

### EXAMPLES

### Example 1

Open-Label Study to Evaluate Motor Improvement after Administration of Modafinil.

A clinical study was performed to evaluate the effect of modafinil on the functional outcome of the paretic arm of patients recovering from a brain injury.

Two chronic post-TBI patients with motor deficits were treated for two consecutive weeks with modafinil (a single 200 mg/day dose, orally). Additionally, three hours after administration of the drug, these patients received one hour of occupational therapy in the morning and one hour of motor training at home in the afternoon. Primary outcomes were the pinch strength measured with a mechanic dynamometer, the number of blocks that the patient can pass in one minute in a box and block test, and the time required to complete a grooved pegboard test as described below.

The Grooved Pegboard test of manual dexterity measures complex visual-motor coordination skill with dominant and non-dominant hands separately by timing how long it takes a subject to place 25 ridged pegs into an equal number of slotted holes angled in different directions. All of the pegs had identical grooves, but the holes were aligned in different positions so that the pegs could only fit in a particular orientation. (Lezak, Neurophysiological Assessment. 3rd ed. (Oxford University Press; New York, 1995.), Trites, Neuropsychological Test Manual (Royal Ottawa Hospital, Ottawa, 1977).)

The “Box and Block Test” is a standardized test of manual dexterity, which provides a baseline for upper extremity manual dexterity and gross motor coordination. The test is quick and simple to administer and uses 150×25 mm (1″) colored wooden blocks.

The Pinch Force Dynamometer is a hand-held device capable of measuring instantaneous strength of the thumb and opposing finger or groupings of fingers as a function of time. The principle components are a pinch force transducer, instrumentation amplifier, and associated cables. Dynamic voltage representing instantaneous finger(s) pinch strength as a function of time is taken from the output of the instrumentation amplifier and sent to a laptop computer or to a data acquisition system for data manipulation, display, correlation with other data sources, and/or storage.

Patients were assessed at baseline, treated for two weeks, assessed at the end of the treatment, and assessed again 90 days after commencement of treatment.

The arms of both the affected and intact arms of subjects were assessed by the three primary outcome measures. As indicated in the charts below, a two-week course of modafinil improved functional motor performance in the paretic hands of the two patients. The improvements were sustained at 90 days. There was also a slight improvement in the intact hand. Neither of the patients reported any adverse effect with the medication. Modafinil seems to be a good stimulant in patients receiving intensive motor rehabilitation programs.

Patient 1

Patient 1 was a female, 27 years old, right-handed. She experienced a severe brain injury at age of 16 (car accident). She remained in coma for more than 2 weeks and progressively recovered consciousness. Two months following the accident she had a severe hemiparesis with significant aphasia. An intensive rehabilitation program enabled her to carry out a practically normal life. Since receiving the modafinil-based rehabilitation program described herein, she became a teacher and studied for a fine arts career. Neurological exam: Alert, oriented in time, space and person. Nominal hypofluent aphasia with preserved repetition. Normal comprehension. Cranial nerves, normal. Motor exam: Revealed normal strength in upper and lower limbs, regular sensitivity and slight increase of right-sided reflex. Normal taxia and normal tone. She failed during transitive gesture but after introducing an object in the action this error was partially corrected with certain clumsiness.

Brain MRI: Lesion in the posterior parietal cortex.

Patient 2

Patient 2 was a 20 year-old male, right-handed. He experienced a severe brain injury at age 18 (car accident). He remained in a coma for more than 2 weeks and progressively recovered consciousness. After two months time, he had a severe hemiparesia that began to revert slowly and progressively. Intensive rehabilitation program allowed him to carry out a practically normal life. After rehabilitation, as described herein, he returned to his studies.

Neurological exam: Alert, oriented in time, space and person. Cranial nerves: strabismus due to involvement of the sixth cranial nerve (external rectus muscle) after accident. Motor exam: Normal strength in upper and lower limbs, sensitivity decreased to inexistent in right upper limb and slight increase in right sided reflex. Normal taxia and normal tone. Dystonic posture of the hand that was corrected with visual feedback. He failed when introducing objects into the action. This was partially corrected afterwards but he showed certain clumsiness. Mirror movements of the normal hand when using the paretic one.

Brain MRI: Left side thalamic lesion.

Neurorehabilitation Regimen

Patients were chronic post-traumatic brain injured individuals that were treated for two consecutive weeks with a 200 mg/day, single dose, of modafinil from Monday through Friday. Additionally, the patients received one hour of occupational therapy three hours after the oral intake of the drug and one hour of motor training at home. Primary outcomes were the pinch strength measured with a mechanic dynamometer, the number of blocks that the patient could pass in one minute in a box and block test, and the time required to complete a grooved pegboard at baseline. Primary outcomes were assessed at two weeks and 90 days from commencement of treatment.

Results

The results for Patients 1 and 2 are provided in Tables 1 and 2, respectively, below.

Patient 1 at baseline (prior to modafinil administration) presented the following primary outcomes in the affected hand: 14.3 pounds in the pinch strength; could pass 34 blocks in one minute in the box and block test; and required 6:02 (min. :sec.) to finalize the grooved pegboard. In the intact hand, patient 1 at baseline showed: 13.5 pounds in the pinch strength; could pass 54 blocks in one minute in the box and block test, and required 1:18 to finalize the grooved pegboard.

At two weeks after commencement of modafinil treatment, patient 1 improved in the affected hand to 16.8 pounds in the pinch strength; could pass 46 blocks in one minute in the box and block test; and required 3:30 (min.:sec.) to finalize the grooved pegboard test. In the intact hand, patient 1 showed 14.6 pounds in the pinch strength; could pass 59 blocks in one minute in the box and block test, and required 1:26 to finalize the grooved pegboard.

At three months in the study, patient 1 showed improvement in the affected hand to 17.6 pounds in the pinch strength; could pass 46 blocks in one minute in the box and block test; and required 3:37 (min.:sec.) to finalize the grooved pegboard test. In the intact hand, patient 1 showed 15 pounds in the pinch strength; could pass 55 blocks in one minute in the box and block test, and required 1:23 to finalize the grooved pegboard test.

Patient 2 at baseline presented in the affected hand: 17 pounds in the pinch strength; could pass 30 blocks in one minute in the box and block test; and required 11:34 to finalize the grooved pegboard. In the intact hand, patient 2 showed 23 pounds in the pinch strength; could pass 62 blocks in one minute in the box and block test, and required 1:18 to finalize the grooved pegboard.

At two weeks, patient 2 improved in the affected hand to 21.3 pounds in the pinch strength; could pass 40 blocks in one minute in the box and block test; and required 6:36 to finalize the grooved pegboard test. In the intact hand, patient 2 showed 24 pounds in the pinch strength; could pass 65 blocks in one minute in the box and block test, and required 1:03 to finalize the grooved pegboard.

At three months into the study, patient 2 presented in the affected hand to 19.3 pounds in the pinch strength; could pass 40 blocks in one minute in the box and block test; and required 5:44 to finalize the grooved pegboard test. In the intact hand, patient 2 showed 23.6 pounds in the pinch strength; could pass 76 blocks in one minute in the box and block test, and required 1:08 to finalize the grooved pegboard test.

TABLE 1
Scores for Patient 1

| | Patient 1 | | | Patient 1 | | |
|---|---|---|---|---|---|---|
| | Affected Hand | | | Intact Hand | | |
| | | 14 | 90 | | 14 | 90 |
| Task | Baseline | Days | Days | Baseline | Days | Days |
| Pinch Strength | 14.3 | 16.8 | 17.6 | 13.5 | 14.6 | 15 |
| (Pounds) | | | | | | |
| Box and Block | 34 | 46 | 46 | 54 | 59 | 55 |
| (# blocks | | | | | | |
| passed in 1 min) | | | | | | |
| Grooved Pegboard | 6:02 | 3:30 | 3:37 | 1:18 | 1:26 | 1:23 |
| (time to complete | | | | | | |
| test) | | | | | | |

TABLE 2
Scores for Patient 2

| | Patient 2 | | | Patient 2 | | |
|---|---|---|---|---|---|---|
| | Affected Hand | | | Intact Hand | | |
| | | 14 | 90 | | 14 | 90 |
| Task | Baseline | Days | Days | Baseline | Days | Days |
| Pinch Strength | 17 | 21.3 | 19.3 | 23 | 24 | 23.6 |
| (Pounds) | | | | | | |
| Box and Block | 30 | 40 | 40 | 54 | 65 | 76 |
| (# blocks | | | | | | |
| passed in 1 min) | | | | | | |
| Grooved Pegboard | 11:34 | 6:36 | 5:44 | 1:18 | 1:03 | 1:08 |
| (time to complete | | | | | | |
| test) | | | | | | |

Conclusion

Orally administered modafinil improved functional motor performance in two patients with motor deficits following traumatic brain injury. The patients did not report any adverse effect from the medication. The data support the use of modafinil to promote effective motor rehabilitation in brain-injured individuals.

All patents, applications, and publications cited in the text above are incorporated herein by reference.

Other variations and embodiments of the invention described herein will now be apparent to those of skill in the art without departing from the disclosure of the invention or the coverage of the claims to follow.

1. A method of treating an impaired neurological function in an individual who has sustained a brain injury comprising administering to said individual an effective amount of modafinil (benzhydrylsulfinylacetamide).

2. The method of according to claim 1, wherein said modafinil is administered to said individual in conjunction with a neurorehabilitation program comprising one or more neurostimuli designed to enhance or restore said impaired neurological function.

3. The method according to claim 2, wherein said neurorehabilitation program provides physical therapy, occupational therapy, speech therapy, and combinations thereof.

4. The method according to claim 2, wherein said neurorehabilitation program is selected from the group consisting of a physical/sensory protocol, an electrical and/or magnetic stimulation regimen, and/or a drug-based stimulation regimen.

5. The method according to claim 4, wherein said physical/sensory protocol comprises a neurostimulus selected from the group consisting of an exercise or task for motor function, an exercise or task for cognitive function, an exercise or task for a combination of motor and cognitive function, a light stimulation, an audio stimulation, a visual stimulation, a tactile stimulation, and combinations thereof.

6. The method according to claim 4, wherein said electrical and/or magnetic stimulation comprises trans-cranial magnetic stimulation (TMS) or deep brain stimulation (DBS)

7. The method according to claim 4, wherein said drug-based stimulation regimen comprises administering a neurostimulant drug.

8. The method according to claim 7, wherein said neurostimulant drug is selected from the group consisting of caffeine, an amphetamine, a dextroamphetamine, a methylphenidate, and combinations thereof.

9. The method according to claim 2, wherein said modafinil is administered to said individual prior to or concurrently with said individual performing an exercise or task to promote or restore an impaired neurological function.

10. The method according to claim 9, wherein administration of modafinil is stopped after said individual performs an exercise or task and wherein said administration is not resumed until further exercise or task is performed.

11. The method according to claim 2, wherein said administration of modafinil and a neurorehabilitation program are ended after a period time, the individual is permitted a period of rest from administration of said modafinil and said neurorehabilitation program, and the individual is then administered modafinil and a neurorehabilitation program for a period of time.

12. The method according to claim 11, wherein after said period of rest, said administration of modafinil is resumed at a different dose and/or said neurorehabilitation program is different from those employed initially.

13. The method according to claim 11, wherein said period of administration of modafinil and a neurorehabilitation program is 2 weeks and said period of rest is 4 to 12 weeks.

14. The method according to claim 1, wherein said modafinil is administered at a dose of from 50 to 600 mg/day.

15. The method according to claim 14, wherein said dose is 100, 200, 400, or 600 mg/day.

16. The method according to claim 15, wherein said dose is 200 mg/day.

17. The method according to claim 2, wherein said modafinil is administered at a dose of from 50 to 600 mg/day in conjunction with administering said neurorehabilitation program.

18. The method according to claim 17, wherein said dose is 100, 200, 400, or 600 mg/day.

19. The method according to claim 18, wherein said dose is 200 mg/day.

20. The method according to claim 1, further comprising administering to said individual a dopaminergic agent that crosses the blood-brain barrier.

21. The method according to claim 1, further comprising administering to said individual a dopaminergic agent selected from the group consisting apomorphine, bromocriptine, amantadine, pergolide, pramipexole, ropinirole, fenoldopam, cabergoline, rotigotine, lysuride, talipexale, 7-OH DPAT, quinpirole, SKF-38393, L-dopa, and combinations thereof.

22. The method according to claim 1, wherein the neurological function impaired in said individual is selected from the group consisting of a cognitive function, a motor function, and a combination of cognitive and motor functions.

23. The method according to claim 1, wherein said brain injury is the result of an event selected from the group consisting of traumatic brain injury, an ischemic episode, spinal cord injury, major organ failure, a brain injury associated with cardiovascular bypass surgery, an anoxic event, a hypoxic event, a drug-induced brain injury, encephalitis, multiple sclerosis, and a degenerative disease.

24. The method according to claim 23, wherein said traumatic brain injury is the result of a fall on a hard surface, a vehicle accident, or a strike to the head.

25. The method according to claim 23, wherein said ischemic event is a stroke.

26-29. (canceled)
